(19) United States
(12) Patent Application Publication
(10) Pub. No.: US 2005/0063998 A1
(43) Pub. Date: Mar. 24, 2005

(54) Oral solution containing galantamine and a sweetening agent

(76) Inventors: Marc Karel Jozef Francois, Kapellen (BE); Tony Mathilde Jozef Kempen, Geel (BE); Eddy Andre Josee De Proost, Turnhout (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/980,970

(22) Filed: Nov. 4, 2004

Related U.S. Application Data
(63) Continuation of application No. 10/111,609, filed on Apr. 25, 2002, now abandoned.

(30) Foreign Application Priority Data
Oct. 26, 1999 (EP) 99203512.1

(51) Int. Cl. A61K 31/55

(52) U.S. Cl. 424/400; 514/214.03

(57) ABSTRACT

The present invention concerns an oral solution comprising galantamine or a pharmaceutically acceptable addition salt thereof; its use and process of preparing the same.

The present invention concerns an oral solution comprising galantamine or a pharmaceutically acceptable addition salt thereof; its use and process of preparing the same.

Galantamine (I), a tertiary alkaloid, has been isolated from the bulbs of the Caucasian snowdrops Galanthus woronowi (Proskurnina, N. F. and Yakoleva, A. P. 1952, Alkaloids of Galanthus woronowi. II. Isolation of a new alkaloid. (In Russian.) Zh. Obschchei Khim. (J. Gen. Chem.) 22, 1899-1902). It has also been isolated from the common snowdrop Galanthus nivalis (Boit, 1954).

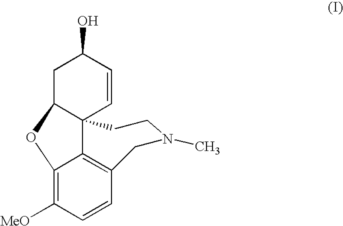

The chemical name of galantamine is [4aS-(4aα, 6β, 8aR*)]-4a, 5,9,10,11,12-hexa-hydro-3-methoxy-11-methyl-6H-benzofuro[3a,3,2-ef][2]benzazepin-6-ol; both the base compound and its hydrobromide are laevorotatory. Galantamine is a well-known acetylcholinesterase inhibitor which is active at nicotinic receptor sites but not on muscarinic receptor sites. It is capable of passing the blood-brain barrier in humans, and presents no severe side effects in therapeutically effective dosages.

Galantamine has been used extensively as a curare reversal agent in anaesthetic practice in Eastern bloc countries (cf. review by Paskow, 1986) and also experimentally in the West (cf. Bretagne and Valetta, 1965: Wislicki, 1967; Consanitis, 1971).

Galantamine has been marketed by Waldheim (Sanochemia Gruppe) as Nivalin™ in Germany and Austria since the 1970s for indications such as facial neuralgia.

The use of galantamine or an analogue or a pharmaceutically acceptable acid addition salt thereof for the preparation of a medicament for treating Alzheimer's Dementia (AD) and related dementias has been described in EP-0,236,684 (U.S. Pat. No. 4,663,318). This patent generically discloses liquid galantamine dosage forms, more in particular oral suspensions or solutions in aqueous ethanol.

EP 0,449,247 generically discloses solutions or suspensions of galantamine or a pharmaceutically acceptable addition salt thereof in organic or inorganic media, such as oils or water, for the treatment of alcoholism. WO 94/16708 generically discloses the same compositions for the treatment of nicotine dependency.

WO 97/26887 describes ocular, oral and parenteral aqueous solutions comprising galantamine or a pharmaceutically acceptable addition salt thereof for the treatment of glaucoma, trisomy or myasthenia gravis. These liquid dosage forms are only generically described.

Oral administration of a liquid galantamine dosage form may offer an attractive way to treat patients suffering from Alzheimer disease and related dementia, vascular dementia, mixed (Alzheimer and vascular) dementia, mild cognitive impairment (MCI), Lewy body disease (LBD), Parkinson disease, schizophrenia, arthritic disorders, chronic fatigue syndrome, facial neuralgia, attention deficit disorders, obstructive sleep apnoea, jet lag, alcohol dependence, nicotine dependence, mania, trisomy, myasthenia gravis, Eaton-Lambert syndrome. The present invention further relates to a method of treating warm-blooded animals suffering from Alzheimer disease and related dementia, vascular dementia, mixed (Alzheimer and vascular) dementia, mild cognitive impairment (MCI), Lewy body disease (LBD), Parkinson disease, schizophrenia, arthritic disorders, chronic fatigue syndrome, facial neuralgia, attention deficit disorders, obstructive sleep apnoea, jet lag, alcohol dependence, nicotine dependence, mania, trisomy, myasthenia gravis, Eaton-Lambert syndrome because of ease of administration. Solid oral dosage forms such as tablets or capsules are not the most suitable dosage forms to treat said conditions since their administration can be a problem (swallow resistance or difficulties). Oral administration is preferred to parenteral administration because the latter is inconvenient and painful and reduces patient's compliance.

Thus, the present invention concerns an oral solution comprising galantamine or a pharmaceutically acceptable addition salt thereof characterized in that it comprises from 0.005 to 3% (w/v) of a sweetening agent.

When being dissolved in an aqueous medium, galantamine exhibits a slightly unpleasant taste. Surprisingly, this unpleasant taste can completely be masked by including a sweetening agent from 0.005 to 3% (w/v; weight based on the total volume of the formulation), preferably from 0.01 to 1% (w/v), more preferably from 0.01 to 0.1% (w/v) and most preferred is 0.05% (w/v). Consequently no additional flavouring agents are required. Suitable sweetening agents are preferably intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, cyclamate, neomate, neohesperidine dihydrochalcone or mixtures thereof, thaumatin, palatinit, stevioside and rebaudioside, sodium saccharin being preferred.

Galantamine or a pharmaceutically acceptable addition salt thereof dissolved in an aqueous medium is most stable in weak acid conditions (pH=±5), whereas it decomposes in acidic and alkaline medium.

For commercial sale, oral solutions are often filled into glass containers. It is a known phenomenon that glass can leach hydroxy ions, affecting in this way the pH and possibly the stability of its contents. It is general practice to assure a stable pH by including buffering agents in formulations, especially when packed in untreated glass containers. However, including extra excipients in a formulation increases the risk of drug-excipients or excipients-excipients interactions. It also increases the risk of adverse side effects experienced by patients taking the medication. From a commercial point of view, it increases the cost of the end product.

When filled in USP type III amber glass bottles, the pH of the present oral solution proved to remain within the preferred shelf-live specification (pH 4-8), without the incorporation of buffering agents. Thus the bulk liquid carrier of the solution of the present invention is a plain aqueous solution, i.e. a non-buffered aqueous solution.

The term ‘bulk liquid carrier’ defines the major part of the solution, preferably ranging from about 70 up to about 99% (w/w; weight based on the total weight of the formulation), more preferably ranging from about 80 up to about 99% (w/w). The water making up the bulk liquid carrier is preferably purified water or demineralized water, purified water being preferred.

By adding suitable pharmaceutically acceptable acids or bases, the pH of the solution of the present invention can be adjusted to range from 4 to 8, preferably from 4 to 6, more preferably from 5 to 6 and most preferred is 5. Suitable pharmaceutically acceptable acids comprise inorganic acids, such as hydrohalic acids, e.g. hydrochloric or hydrobromic acid, sulfuric, nitric, phosphoric and the like acids, or organic acids, such as, for example, acetic, propanoic, hydroxyacetic, lactic, pyruvic, oxalic, malonic, succinic, maleic, fumaric, malic, tartaric, citric, ascorbic and the like acids. Appropriate bases comprise organic and inorganic bases, for example ammonium acetate, ammonia, alkali or earth alkaline metal hydroxides, sodium carbonate, sodium hydrogen carbonate, sodium phosphate and the like.

In order to increase the shelf life of the present solution, which is likely to be used repeatedly, the growth of micro-organisms such as bacteria, yeasts and fungi in the formulation may be prevented by adding one or more preservatives. Pharmaceutically acceptable preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, chlorocresol, phenol, phenyl ethyl alcohol, organic acids or salts and derivatives thereof such as benzoic acid, sodium benzoate, sorbic acid, potassium sorbate, parabens such as methyl parahydroxybenzoate or propyl parahydroxybenzoate, aqua conservans; phenylmercuri nitrate,-borate,-acetate; chloorhexidine diacetate,-digluconate. The formulation may also contain anti-oxydants, such as, for example, sodium meta-bisulfite, sodium bisulfite, sodium sulfite, sodium thiosulfate, ascorbic acid, or complex forming agents such as EDTA, citric acid, tartaric acid, sodium-hexametaphosphate and the like. The concentration of the preservative will range from 0% to 2% (w/w), depending on the actual preservative being used. Preferable preservatives in the composition of the present invention are paraben preservatives, more in particular a mixture of methyl parahydroxybenzoate and propyl parahydroxybenzoate. The concentration of the anti-oxydants generally amounts up to 0.2% (w/v) and the amount of complex forming agents up to 3% (w/v).

Although a flavouring agent is not required to mask the unpleasant taste of galantamine in the present solution, one or more flavouring substances may optionally be added to the subject invention to further optimize its palatability. Suitable flavouring substances are fruit flavours such as cherry, raspberry, black currant or strawberry flavour, or stronger flavours, such as Caramel Chocolate flavour, Mint Cool flavour, Fantasy flavour and the like. Combinations of flavours are advantageously used. The total concentration of the flavouring substances may range from 0.01% to 0.5%, preferably from 0.03% to 0.2% and most preferably from 0.05% to 0.1%.

The oral solution of the present invention may also optionally include viscosity regulating agents, for example, alkylcelluloses such as methylcellulose; hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectines such as sodium carboxymethylamylopectine; chitin derivates such as chitosan; di-, oligo- and polysaccharides such as trehalose, cyclodextrins and derivatives thereof, alginic acid, alkali metal and ammonium salts thereof, carrageenans, galactomannans, tragacanth, agar-agar, gummi arabicum, guar gummi and xanthan gummi; polyacrylic acids and the salts thereof; polymethacrylic acids, the salts and esters thereof, methacrylate copolymers; polyvinylalcohol; polyvinylpyrrolidone or copolymers thereof; polyalkylene oxides such as polyethylene oxide and polypropylene oxide and copolymers of ethylene oxide and propylene oxide.

An interesting composition according to the present invention comprises by weight on the total volume of the composition:

Galantamine or

| | |
|---|---|
| a pharmaceutically acceptable addition salt thereof | 0.1 to 2% |
| Preservative(s) | 0 to 2% |
| Intense sweetener | 0.005 to 3% |
| Acid or base | q.s.* ad pH 4-8 |
| Purified water | q.s.* ad 100%. |

*q.s. ad = quantum satis ad = as much as needed up to.

The present invention also relates to a process of preparing the oral solution of the present invention comprising the steps of:
mixing galantamine or a pharmaceutically acceptable addition salt thereof, intense sweetener, optionally other pharmaceutically acceptable excipients and bulk liquid carrier until complete dissolution;
optionally adjusting the pH of the resulting solution to pH 4-8;
diluting the resulting solution to the desired end-volume with purified water.

The above general route of preparing the oral solution of the present invention may be modified by a person skilled in the art by for instance adding certain ingredients at other stages than indicated above. For example, the intense sweetener can first be dissolved followed by dissolving galantamine.

A further aspect of the present invention concerns the use of the above formulation as a medicine, especially the use for the manufacture of a medicament for treating patients suffering from Alzheimer disease and related dementia, vascular dementia, mixed (Alzheimer and vascular) dementia, mild cognitive impairment (MCI), Lewy body disease (LBD), Parkinson disease, schizophrenia, arthritic disorders, chronic fatigue syndrome, facial neuralgia, attention deficit disorders, obstructive sleep apnoea, jet lag, alcohol dependence, nicotine dependence, mania, trisomy, myasthenia gravis, Eaton-Lambert syndrome. The present invention further relates to a method of treating warm-blooded animals suffering from Alzheimer disease and related dementia, vascular dementia, mixed (Alzheimer and vascular) dementia, mild cognitive impairment (MCI), Lewy body disease (LBD), Parkinson disease, schizophrenia, arthritic disorders, chronic fatigue syndrome, facial neuralgia, attention deficit disorders, obstructive sleep apnoea, jet lag, alcohol dependence, nicotine dependence, mania, trisomy, myasthenia gravis, Eaton-Lambert syndrome by administering to said warm-blooded animals an therapeutically effective amount of the oral solution of the present invention.

The daily required dosage of galantamine or a pharmaceutically acceptable addition salt thereof, the amount per single dose and the frequency of dosing varies with the condition being treated, the severity of said condition, and the patient being treated. The daily dosage may range from 5 to 1000 mg, preferably from 5-45 mg, more preferably from 10-35 mg and most preferred from 15-25 mg.

Experimental Part

(a) Composition

| | |
|---|---|
| Galantamine hydrobromide | 5.124 mg (4 mg of galantamine base) |
| Methyl parahydroxybenzoate | 1.800 mg |
| Propyl parahydroxybenzoate | 0.200 mg |
| Sodium saccharin dihydrate | 0.500 mg |
| Sodium hydroxide | q.s.* ad pH 4.9-5.1 |
| Purified water | q.s.* ad 1.0 ml. |

*q.s. ad = quantum satis ad = as much as needed up to.

(b) Preparation of a 300 l Batch

150 l of purified water was transferred into a stainless steel liquid processor and was heated up to 45-50° C., while stirring. Methyl parahydroxybenzoate (0.54 kg) and propyl parahydroxybenzoate (0.06 kg) were added and the resulting mixture was stirred until complete dissolution. 135 l of purified water was added and the whole was stirred until homogeneous and cooled down to 20-30° C. Galantamine hydrobromide was added and the mixture was stirred until complete dissolution. Sodium saccharine dihydrate was added and the whole was mixed until complete dissolution. A 0.1 N aqueous sodium hydroxide solution was added to adjust the pH of the solution to 4.9-5.1. Purified water was added to adjust the total volume to 300 l, while mixing until homogeneous. The final solution was filtered over a 25 μm polypropylene filter.

(c) Stability of a Galantamine Hydrobromide Aqueous Solution as a Function of pH

A galantamine hydrobromide 10 mg/ml aqueous solution was stored in acid, neutral and alkaline conditions at 80° C. for 1 up to 24 hours. After storage, the solutions were analyzed using HPLC for the presence of degradation products. The table below shows the obtained results.

| Medium | Storage | Degradation compounds |
|---|---|---|
| Acid (1 N hydrochloric acid) | 1 hour | 18.4% |
| Neutral (water pH 5.2) | 24 hours | No degradation detected |
| Alkaline (1 N sodium | 24 hours | 0.2% |
| hydroxide) | | |

Galantamine hydrobromide remained stable in the aqueous medium of pH 5.2 while it decomposed in acidic and alkaline medium.

(d) pH Stability Study of the Present Solution when Filled in Glass Bottles

The galantamine hydrobromide solution as described under point (a) was filled into 100 ml USP type III amber glass bottles and stored at different conditions. The pH of the solution was determined after predetermined time intervals. The table below gives an overview of the obtained pH values under different conditions.

| Storage condition | Time (months) | pH |
|---|---|---|
| 4° C. | 3 | 5.5 |
| 25° C./60% RH* | 1 | 5.5 |
| | 3 | 5.6 |
| | 6 | 5.7 |
| | 9 | 5.5 |
| | 12 | 5.6 |
| 30° C./≦40% RH | 3 | 5.6 |
| | 6 | 5.7 |
| | 9 | 5.6 |
| | 12 | 5.6 |
| 40° C. | 1 | 5.7 |
| | 3 | 5.6 |
| | 6 | 5.6 |
| 50° C. | 1 | 5.7 |
| | 3 | 5.6 |
| light | 0.3 days | 5.5 |

*RH = Relative Humidity

1. An oral solution comprising:
(a) galantamine or a pharmaceutically acceptable addition salt thereof;
(b) a bulk liquid carrier; and
(c) 0.005 to 3 % (w/v) of a sweetening agent;
wherein said bulk liquid carrier is an aqueous solution comprising from about 70 to about 99% (w/w; weight based on the total weight of the solution) of water, and wherein the sweetening agent masks the unpleasant taste of the galantamine or pharmaceutically acceptable addition salt thereof.

2. An oral solution according to claim 1 wherein the bulk liquid carrier is a non-buffered aqueous solution.

3. An oral solution according to claim 1 wherein the pharmaceutically acceptable addition salt of galantamine is galantamine hydrobromide.

4. An oral solution according to claim 1 wherein the concentration of the sweetening agent ranges from 0.01 to 1% (w/v).

5. An oral solution according to claim 4 wherein the sweetening agent is sodium saccharin dihydrate.

6. An oral solution according to claim 1 wherein the pH of the solution is from 4 to 8.

7. (Canceled)

8. A method of treating a patient suffering a disease, comprising the steps of:
(a) preparing the oral solution of claim 1; and
(b) administering the oral solution to the patient.

9. The method of claim 8, wherein said disease is selected from the group consisting of Alzheimer disease and related dementia, vascular dementia, mixed (Alzheimer and vascular) dementia, mild cognitive impairment (MCI), Lewy body disease (LBD), Parkinson disease, schizophrenia, arthritic disorders, chronic fatigue syndrome, facial neuralgia, attention deficit disorders, obstructive sleep apnoea, jet lag, alcohol dependence, nicotine dependence, mania, trisomy, myasthenia gravis, and Eaton-Lambert syndrome.

10. A process of preparing an oral solution as defined in claim 1 comprising the steps of:
(a) completely dissolving galantamine or a pharmaceutically acceptable addition salt thereof, intense sweetener, and optionally other pharmaceutically acceptable excipients in a bulk liquid carrier to form a solution;
(b) optionally adjusting the pH of the resulting solution to pH 4-8; and
(c) diluting the solution to the desired end-volume with purified water.

11. An oral solution according to claim 1 having the following composition

| | |
|---|---|
| Galantamine hydrobromide | 5.1 mg; |
| Methyl parahydroxybenzoate | 1.8 mg; |
| Propyl parahyoxybenzoate | 0.2 mg; |
| Sodium saccharin dihydrate | 0.5 mg; |
| Sodium hydroxide | q.s. ad pH 4.9-5.1; and |
| Purified water | q.s. ad 1 mL. |
